(19) United States
(12) Patent Application Publication
(10) Pub. No.: US 2020/0170971 A1
(43) Pub. Date: Jun. 4, 2020

(54) DISULFIDE-MASKED PRO-CHELATOR COMPOSITIONS AND METHODS OF USE

(71) Applicant: ARIZONA BOARD OF REGENTS ON BEHALF OF THE UNIVERSITY OF ARIZONA, Tucson, AZ (US)

(72) Inventors: Elisa Tomat, Tucson, AZ (US); Eman A. Akam, Tucson, AZ (US)

(21) Appl. No.: 16/632,217

(22) PCT Filed: Jul. 17, 2018
(86) PCT No.: PCT/US18/42458

§ 371 Date: Jan. 17, 2020

Related U.S. Application Data
(60) Provisional application No. 62/533,964, filed on Jul. 18, 2017.

(51) Int. Cl. A61K 31/175; A61P 35/00

(52) U.S. Cl. CPC A61K 31/175; A61P 35/00

(57) ABSTRACT

Pro-chelator compositions featuring disulfide masks that upon activation yield active chelators. The pro-chelator compositions may be activated intracellularly, for example within cells featuring metal ion dysregulation, cells that proliferate abnormally, etc. The pro-chelators of the present invention include thiosemicarbazones, semicarbazones, and aroyl hydrazones. The pro-chelator compositions of the present invention may be used for a variety of purposes including inhibiting cell proliferation, or treating conditions associated with metal ion dysregulation or abnormal cell proliferation.

### CROSS REFERENCE

This application claims priority to U.S. Provisional Patent Application No. 62/533,964, filed Jul. 18, 2017, the specifications of which are incorporated herein in their entirety by reference.

### FIELD OF THE INVENTION

The present invention relates to chelating compounds, more particularly to pro-chelators that upon activation yield active chelators, such as but not limited to disulfide-masked pro-chelators. The pro-chelators of the present invention may be used for intracellular metal sequestration or for other purposes.

### BACKGROUND OF THE INVENTION

The present invention features pro-chelator compositions, e.g., pro-chelator molecules, which upon activation yield an active chelator. For example, a disulfide reduction/activation switch incorporated on thiosemicarbazone scaffolds results in activated iron prochelators. In addition to thiosemicarbazone pro-chelators, the present invention also features several tridentate donor sets including aroyl hydrazones and semicarbazones.

As described in Example 1, the compositions of the present invention have been found to have anti-proliferative effects in certain breast adenocarcinoma cells lines (MCF7 and metastatic MDA-MB-231), and they do not result in the intracellular generation of oxidative stress. Flow cytometry experiments in cultured Jurkat cells indicated that the tested prochelators lead to cell cycle arrest at the G_{1/0} interface and result in induction of apoptosis. Thus, the present invention also features methods of reducing or inhibiting proliferation of cells that are in an abnormal proliferative state (e.g., cancer cells), methods for inducing apoptosis in such cells, methods of treating cancers, methods of treating diseases or conditions associated with metal ion dysregulation, etc.

### SUMMARY OF THE INVENTION

The present invention features pro-chelators comprising at least one pro-ligand and a disulfide bond, having the disulfide bond connected to the pro-ligand, and having each pro-ligand comprise at least two donor atoms. In some embodiments, the pro-chelator is selectively reduced within a cell with iron ion dysregulation. The pro-chelator compositions of the present invention may be used for biological purposes, e.g., for intracellular metal ion chelation (e.g., iron chelation).

The present invention also features a method of reducing or inhibiting proliferation of a cell. In some embodiments, the method comprises introducing a pro-chelator of the present invention (e.g., according to Formula I, Formula II, Formula III). The pro-chelator can be activated in the cell yielding an active chelator. The active chelator can chelate metal ions and reduce or inhibits proliferation of the cell. In one embodiment, the cell is a cell with iron dysregulation and the active chelator chelates iron ions. In another embodiment, the cell is proliferating abnormally, and the active chelator chelates iron ions.

The present invention also features a method of treating a clinical condition associated with metal ion dysregulation in a subject in need of said treatment. The method may comprise administering to the subject a therapeutically effective amount of a pro-chelator of the present invention. The pro-chelator is reduced within a cell having a metal ion dysregulation to yield an active chelator that chelates metal ions. Chelation of metal ions by the active chelator within the cell with a metal dysregulation alleviates the clinical condition associated with metal ion dysregulation. In some embodiments, the clinical condition associated with metal ion dysregulation is a cancer.

The present invention also features a method of treating a clinical condition associated with abnormal cell proliferation in a subject in need of said treatment. The method may comprise administering to the subject a therapeutically effective amount of a pro-chelator of the present invention. The pro-chelator is reduced within a cell that proliferates abnormally to yield an active chelator that chelates metal ions. Chelation of metal ions by the active chelator within the cell that proliferates abnormally alleviates the clinical condition associated with abnormal cell proliferation. In some embodiments, the clinical condition associated with abnormal cell proliferation is a cancer.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1 shows prior art examples of iron-binding units in chelators employed for biomedical applications.

FIG. 2 shows an example of a scheme for reduction/activation of a disulfide switch in thiosemicarbazone pro-chelator (TC1-S)₂.

FIG. 3A shows non-limiting examples of thiosemicarbazone pro-chelators.

FIG. 3B shows non-limiting examples of semicarbazone pro-chelators.

FIG. 3C shows non-limiting examples of aroyl hydrazone pro-chelators.

FIG. 4 shows IC50 values for disulfide pro-chelator compositions in the cancer cell lines MDA-MB-231 and MCF-7 and the normal cell line MRC5 after 72-hour incubations with indicated compound. Values were determined using standard MTT assay.

FIG. 5 shows an assessment of intracellular generation of ROS upon incubation with selected pro-chelators. MDA-MB-231 cells were treated with the indicated compounds (50 μM, 2 h), washed, treated with DCFH₂-DA (30 μM, 30 min) in PBS, and then analyzed by flow cytometry. Hydrogen peroxide is used as a positive control and SIH as a negative control. Values are presented as mean ±SDM (n=3), **p<0.01.

FIG. 6 shows effects of pro-chelators on cell cycle in cultured Jurkat cells. Cells were treated with the compounds (10 or 50 μM, 12 h), harvested, fixed, pelleted and then treated with RNAse and propidium iodide (0.5 mg/mL and 40 μg/mL, respectively, 30 min) prior to analysis by flow cytometry. Values are presented as mean ±SDM (n=3), *p<0.05 and **p<0.01.

FIG. 7 shows an investigation of cell death in the presence of disulfide-based pro-chelators. Jurkat cells were incubated with the tested compounds (20 μM, 48 h) or vehicle only (DMSO, untreated control). Following treatment with FTIC-Annexin V (AnnV) and propidium iodide (PI), the cells were analyzed by flow cytometry. Values are presented as mean ±SDM (n=3), *p<0.05 and **p<0.01.

FIG. 8 shows Scheme 1, which is a non-limiting example of synthesis of disulfide-based prochelators via condensation of a thiosemicarbazide, semicarbazide, or hydrazide with a 2,2′-dithiodibenzyl dicarbonyl precursor.

### DEFINITIONS

“Donor atoms” refers to atoms which comprise at least one pair of electrons which can be donated to coordinate a metal atom or ion. “Polydentate” refers to a ligand attached to the central atom in a coordination complex by two or more bonds. “Solubilizing” refers to a group which increases or decreases a hydrophilicity of a molecule to increase its solubility in an environment. “Biologically active” refers to a group which is capable of exerting a pharmacological activity on a biological sample.

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tert-butyl, pentyl, and the like.

“Aryl” refers to a monovalent mono-, bi- or tricyclic aromatic hydrocarbon moiety of 6 to 15 ring atoms that is optionally substituted with one or more substituents within the ring structure. When two or more substituents are present in an aryl group, each substituent is independently selected.

The term “heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring is optionally substituted independently with one or more substituents. Exemplary heteroaryls include, but are not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

“Pharmaceutically acceptable excipient” refers to an excipient that is useful in preparing a pharmaceutical composition that is generally safe and non-toxic. The excipient may be acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The terms “pro-drug” and “prodrug” are used interchangeably herein and refer to a pharmacologically substantially inactive derivative of a parent drug molecule that requires biotransformation, either spontaneous or enzymatic, within the organism to release the active drug. Prodrugs are variations or derivatives of the compounds of this invention, which have groups cleavable under metabolic conditions. Prodrugs become the compounds of the invention that are pharmaceutically active in vivo when they undergo solvolysis or reduction or other reaction eliciting activation under physiological conditions or undergo enzymatic processing. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives that are well known to one skilled in the art, such as, but not limited to, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. For example, a compound of the invention having free amino, amido, hydroxy or carboxylic groups can be converted into prodrugs. Prodrugs include compounds wherein an amino acid residue, or a polypeptide chain of two or more (e.g., two, three or four) amino acid residues, which are covalently joined through peptide bonds to free amino, hydroxy or carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, omithine and methionine sulfone. Prodrugs also include compounds wherein carbonates, carbamates, amides and alkyl esters that are covalently bonded to the above substituents of a compound of the invention through the carbonyl carbon prodrug side chain.

The term “chelator” refers to a compound or a moiety that is capable of coordinating (or binding) a metal ion in a polydentate (e.g., coordination via two or more atoms of moieties) fashion. The terms “pro-chelator” and “prochelators” are used interchangeably herein and refer to a compound or a moiety that is transformed into a chelator following activation via a chemical reaction (e.g., with or by another compound including via redox reaction) or by an enzyme.

The term “anion stabilizing group” refers to a moiety whose presence in the molecule increases the stability of the anion relative to the absence of such a group. One skilled in the art can readily determine whether a substituent or a moiety is an anion stabilizing group, e.g., by determining the increase in the acidity of the resulting compound compared to a corresponding compound in the absence of such group. One can empirically determine the anion stabilization of a particular group by determining the pKa of the compound having the anion stabilizing group and comparing it with the pKa of the corresponding compound in the absence of the anion stabilizing group. Typically, the anion stabilizing compound will have a lower pKa, i.e., it will be more acidic.

“Protecting group” refers to a moiety, except alkyl groups, that when attached to a reactive group in a molecule masks, reduces or prevents that reactivity. Examples of protecting groups can be found in T.W. Greene and P.G.M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “iron ion-overload” (or iron dysregulation) refers to a cell whose iron ion concentration is above the normal iron ion concentration and manifest abnormal clinical condition. The iron ion-overload can be the cause of the clinical condition or it can be a result of the clinical condition. It should be appreciated that the iron ion concentration of a normal cell can vary depending on the type of cells. The terms “iron overload” and “iron ion-overload” are used interchangeably herein and typically refers to the condition of patients presenting systemic iron concentrations that are significantly higher than normal, e.g., the amount of iron concentration in subjects that do not show any observable clinical condition(s). Iron overload can be due to accidental exposure to excessive iron or to genetic conditions that lead to accumulation of iron, such as hemochromatosis, or to conditions that require multiple blood transfusions, such as thalassemia. Thus, a clinical condition associated with the iron overload includes clinical condition in which the iron ion-overload is a cause or the effect of such a clinical condition.

It is believed that cancer cells do not have a significantly higher systemic iron levels in general. However, cancer cells are more susceptible to iron deprivation because they require higher iron levels. Accordingly, in some embodiments of the invention, the method includes treating a cancer patient by administering a therapeutically effective amount of a compound of the invention. In these embodiments, the amount of compound administered is of sufficient amount to cause deprivation of iron in cancer cells to effectively cause apoptosis of cancer cells.

### DETAILED DESCRIPTION OF THE INVENTION

The present invention features pro-chelator compositions, e.g., pro-chelator molecules, which upon activation yield active chelators. Pro-chelators of the present invention may comprise a disulfide mask, wherein a reduction/activation switch incorporated in the pro-chelators (e.g., thiosemicarbazone) results in active iron prochelators. The present invention also features several tridentate donor sets including aroyl hydrazones and semicarbazones.

In one embodiment, the present invention features a pro-chelator comprising at least one pro-ligand and a disulfide bond, having the disulfide bond connected to the pro-ligand, and having each pro-ligand comprise at least two donor atoms. In another embodiment, the present invention features a method of preventing iron-deficiency anemia while treating a subject having malignant cells characterized by a reprogrammed iron metabolism. As a non-limiting example, the method may comprise: providing a pro-chelator to a bloodstream of the subject; and transporting the pro-chelator to an intracellular space of a malignant cell of the subject. In preferred embodiments, an active chelator may be selectively released from the pro-chelator by reduction of the disulfide bond within the intracellular space. In additional preferred embodiments, the active chelator may coordinate Fe selectively within the intracellular space to form a metal complex, and not coordinate Fe in the bloodstream of the subject. In further preferred embodiments, the selective coordination of Fe may be effective for treating the malignant cells without causing iron-deficiency anemia.

According to some embodiments, the pro-chelator may comprise two pro-ligands, connected by the disulfide bond. According to some other embodiments, the pro-chelator may comprise one pro-ligand and a solubilizing or biologically active moiety, connected by the disulfide bond. In some preferred embodiments, the pro-chelator may be activated to transform each pro-ligand to an active bidentate, tridentate, or polydentate chelator. As a non-limiting example, the pro-chelator may be activated by reduction of the disulfide bond.

In other embodiments, each active chelator may comprise a semicarbazone, a thiosemicarbazone, a hydrazone, or a thiohydrazone moiety. In still other embodiments, each active chelator may comprise an iminic position, and may comprise an electron-withdrawing group at the iminic position. In yet other embodiments, each active chelator may be configured to coordinate Fe to form a metal complex. In even another embodiment, the metal complex has a Fe^{III}/Fe^{II} potential of about −200 to 200 mV compared to a Normal Hydrogen Electrode (NHE). In other embodiments, the metal complex has a Fe^{III}/Fe^{II} potential of about −1000 to 1000, −900 to 900, −800 to 800, −700 to 700, −600 to 600, −500 to 500, −400 to 400, −300 to 300, −100 to 100, −50 to 50, or −10 to 10 mV, compared to a Normal Hydrogen Electrode (NHE).

In another embodiment, the pro-chelator may comprise a structure according to Formula II, Formula III, Formula IV, or Formula V.

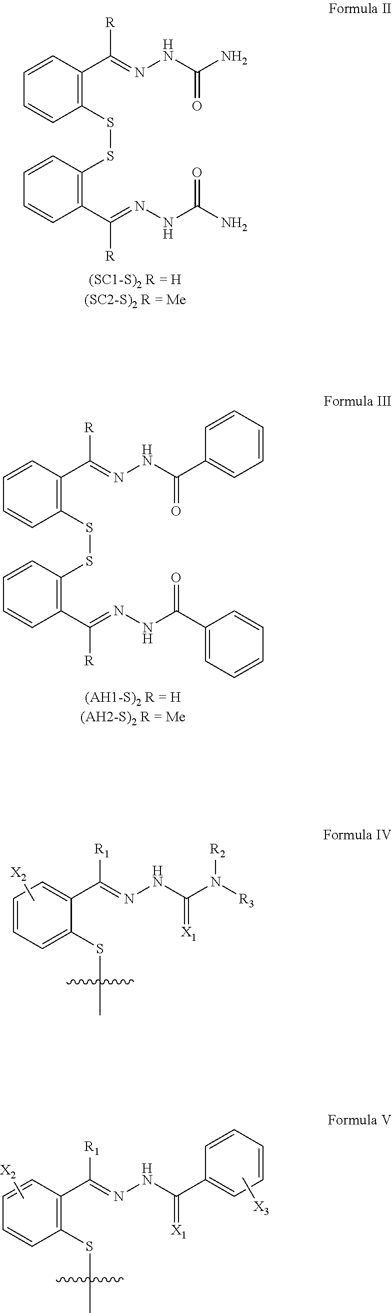

In an embodiment, the present invention may feature a pro-chelator according to Formula II or Formula III, wherein R is H, alkyl, aryl, or a derivative thereof. In some embodiments, R may be trifluoromethyl. In other embodiments, the pro-chelator may be redox-activated. In further embodiments, the pro-chelator may be selectively reduced within a cell with iron ion dysregulation.

In another embodiment, the present invention may feature a pro-chelator according to Formula IV. In one embodiment, if X₁=O then R₁=Ph, pyridyl, p-CF₃-Ph, p-NO₂-Ph, CCl₃, or CF₃; X₂=H, alkyl, alkoxy, halo, CF₃, or NO₂; R₂=H, alkyl, aryl, or substituted aryl; and R₃=H, alkyl, aryl, or substituted aryl. In an alternative embodiment, if X₁=S, then R₁=Ph, pyridyl, p-CF₃-Ph, p-NO₂-Ph, CCl₃, or CF₃; X₂=H, alkyl, alkoxy, halo, CF₃, or NO₂; R₂=alkyl, aryl, or substituted aryl; and R₃=alkyl, aryl, or substituted aryl.

In still another embodiment, the present invention may feature a pro-chelator according to Formula V. In some embodiments, X₁=O or S; R₁=Ph, pyridyl, p-CF₃-Ph, p-NO₂-Ph, CCl₃, or CF3; X₂=H, alkyl, alkoxy, halo, CF₃, or NO₂; and X₃=H, alkyl, alkoxy, halo, CF₃, or NO₂.

In one embodiment, the present invention features a method of reducing or inhibiting proliferation of a cell. As a non-limiting example, the method may comprise introducing a pro-chelator, wherein the pro-chelator is activated by reduction of the disulfide bond in an intracellular space of the cell to transform each pro-ligand to an active bidentate, tridentate, or polydentate chelator, wherein each active chelator coordinates metal ions and reduces or inhibits proliferation of the cell. In another embodiment, the cell may be a cell with iron dysregulation or a cell that is proliferating abnormally. In still another embodiment, the active chelator may coordinate iron ions.

In some embodiments, the present invention may feature a method of treating a clinical condition associated with metal ion dysregulation in a subject in need of said treatment. As a non-limiting example, the method may comprise administering to the subject a therapeutically effective amount of a pro-chelator, wherein said pro-chelator is activated by reduction of the disulfide bond in an intracellular space of the cell to transform each pro-ligand to an active bidentate, tridentate, or polydentate chelator that coordinates metal ions, whereby coordination of metal ions by said active chelators alleviates said clinical condition associated with metal ion dysregulation. In one embodiment, the clinical condition associated with metal ion dysregulation may be cancer.

In still other embodiments, the present invention may feature a method of treating a clinical condition associated with abnormal cell proliferation in a subject in need of said treatment. As a non-limiting example, the method may comprise: administering to the subject a therapeutically effective amount of a pro-chelator, wherein said pro-chelator is activated by reduction of the disulfide bond in an intracellular space of the cell to transform each pro-ligand to an active bidentate, tridentate, or polydentate chelator that coordinates metal ions, whereby coordination of metal ions by said active chelators alleviates said clinical condition associated with abnormal cell proliferation. In some embodiments, the clinical condition associated with abnormal cell proliferation may be cancer.

Formula 1 features a pro-chelator of the present invention (a thiosemicarbazone pro-chelator). In some embodiments, R₁ is H or methyl or a derivative thereof. In some embodiments, R₂ is H, methyl, phenyl, other aryl or heteroaryl, or a derivative thereof. In some embodiments, R₁ is H and R₂ is phenyl. In some embodiments, R₁ is H and R₂ is H. In some embodiments, R₁ is methyl and R₂ is phenyl. In some embodiments, R₁ is methyl and R₂ is H. In some embodiments, R₁ is H and R₂ is methyl. In some embodiments, R₁ is methyl and R₂ is an aryl or heteroaryl.

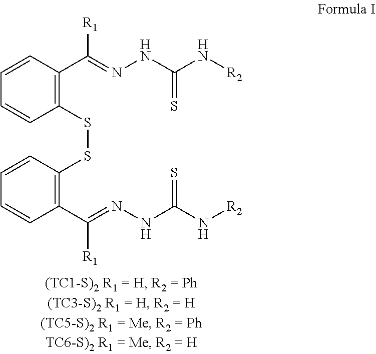

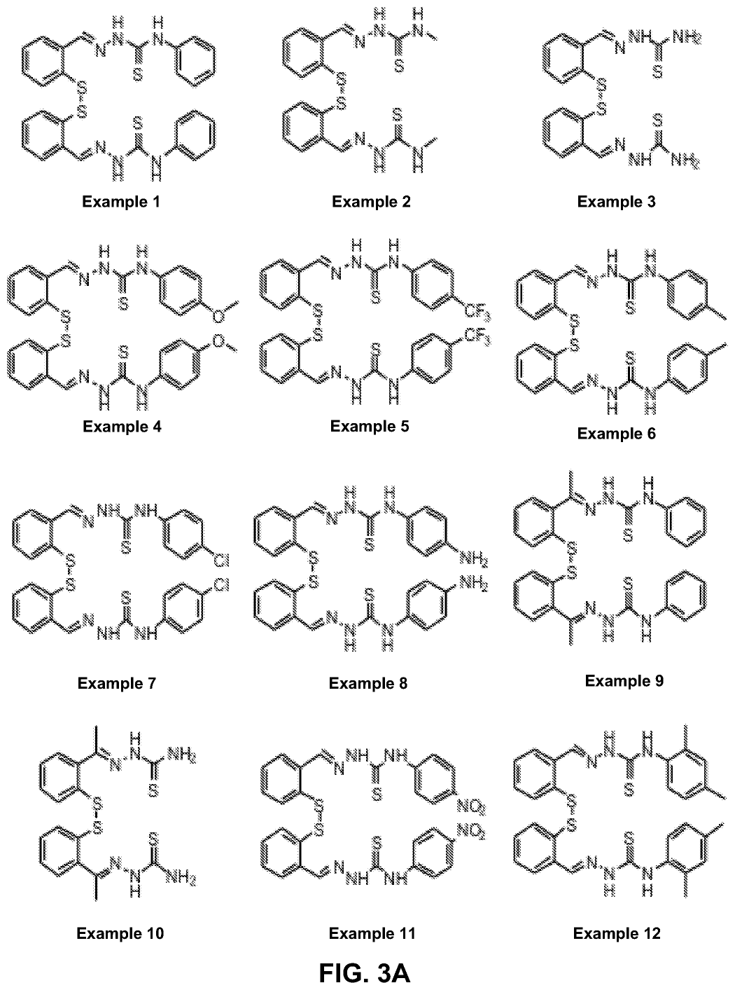

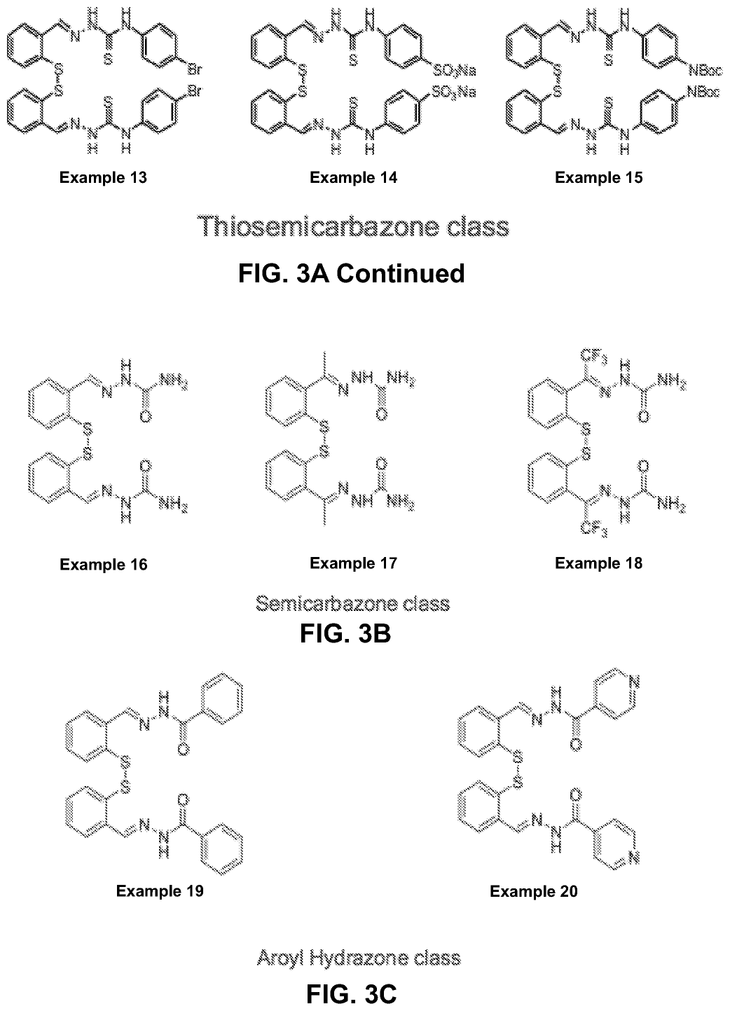

FIG. 3A shows specific examples of Formula 1 (thiosemicarbazone pro-chelator compositions). For example, Example Compound 1 is formed when R₁ is H and R₂ is phenyl. Example Compound 2 is formed when R₁ is H and R₂ is methyl. Example Compound 3 is formed when R₁ is H and R₂ is H. Example Compound 4, Example Compound 5, Example Compound 6, Example Compound 7, Example Compound 8, Example Compound 11, Example Compound 12, Example Compound 13, Example Compound 14, and Example Compound 15 are formed when R₁ is H and R₂ is a phenyl derivative. Example Compound 9 is formed when R₁ is methyl and R₂ is a phenyl derivative. Example Compound 10 is formed when R₁ is methyl and R₂ is H.

Formula II shows semicarbazone pro-chelators. In some embodiments, R is H or methyl or a derivative thereof.

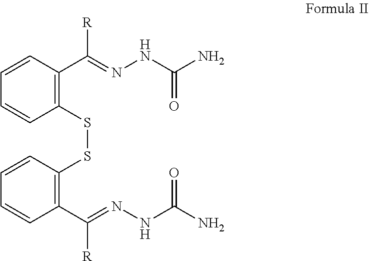

FIG. 3B shows specific examples of Formula II (semicarbazone pro-chelator compositions). For example, Example Compound 16 is formed when R is H. Example Compound 17 is formed when R is methyl. Example Compound 18 is formed when R is a methyl derivative, specifically trifluoromethyl.

Formula III shows aroyl hydrazone pro-chelators. In some embodiments, R is H or methyl or a derivative thereof.

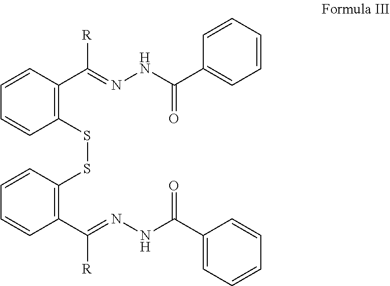

FIG. 3C shows specific examples of Formula III (aroyl hydrazone pro-chelator compositions). For example, Example Compound 19 is formed when R is H. Example Compound 20 is formed when R is methyl and the terminal phenyl groups comprise nitrogen.

The pro-chelator compositions of the present invention may be used for biological purposes, e.g., for intracellular metal ion chelation (e.g., iron chelation). The present invention also features methods of reducing or inhibiting proliferation of cells that are in an abnormal proliferative state (e.g., cancer cells) using the pro-chelator compositions of the present invention. The present invention also features methods for inducing apoptosis in cells that are in an abnormal proliferative state (e.g., cancer cells) using the pro-chelator compositions of the present invention. The present invention also features methods of treating diseases associated with metal ion dysregulation using the pro-chelator compositions of the present invention. The present invention also features methods of treating diseases associated with cells in an abnormal proliferative state (e.g., cancers) using the pro-chelator compositions of the present invention. The present invention also features methods of synthesis of pro-chelators of the present invention.

The present invention also includes pharmaceutical compositions comprising at least one compound of the invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

Non-limiting examples of pharmaceutical carriers and their formulations are described in Remington: The Science and Practice of Pharmacy 1995, edited by E. W. Martin, Mack Publishing Company, 19th edition, Easton, Pa.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation.

### EXAMPLE 1

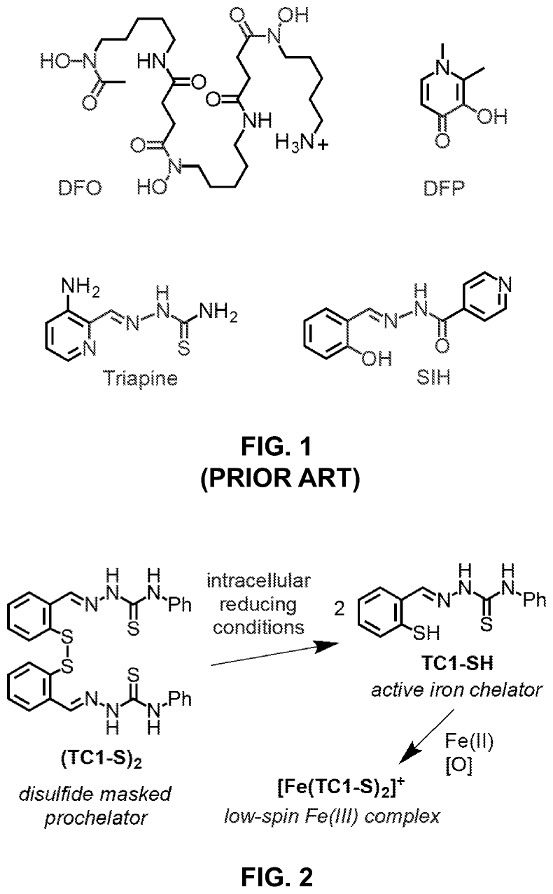

Rapidly dividing malignant cells are characterized by a reprogrammed iron metabolism, which enhances intracellular iron availability through an altered expression of several key proteins for iron homeostasis. Correspondingly, the expression levels of transferrin receptors, ferroportin and ferritin have been identified as prognostic markers in breast cancer patients. At a molecular level, reactivity-based fluorescent probes of intracellular iron have shown recently that the labile iron pool is larger in several cancer cell lines when compared to non-malignant ones. As such, high-affinity scavengers (chelators) can be employed to target the increased iron needs of cancer cells for the development of antineoplastic agents. The scaffolds of chelators studied in this context vary substantially (see FIG. 1): from hexadentate hydroxamate-based siderophores (e.g., desferrioxamine aka DFO) to bidentate deferiprone (DFP) to tridentate thiosemicarbazones (e.g., Triapine) and aroylhydrazones (e.g., salicyl isonicotinoyl hydrazone aka SIH).

Thiosemicarbazones were found to have growth inhibition effects in sarcomas. Since then, numerous thiosemicarbazones and hydrazones have been studied for their antineoplastic effects in vitro and in animal models.

Furthermore, Triapine has been investigated in several clinical trials. More recently, new classes of thiosemicarbazones were investigated for their antineoplastic effects. These studies produced highly potent compounds such as di-2-pyridylketone 4,4-dimethyl-3-thiosemicarbazone (Dp44mT) and di-2-pyridylketone 4-cyclohexyl-4-methyl-3-thiosemicarbazone (DpC), which proved effective in vitro and in vivo through oral and intravenous administration, and demonstrated the potential to overcome multidrug resistance, as well as some of the limitations of Triapine including methemoglobin formation. DpC has entered clinical trials in 2016.

Within the study of metal-binding pharmaceuticals, prochelation strategies are being pursued to minimize off-target toxicity through inclusion of structural motifs that render the chelator inactive until triggered by disease-specific conditions. Examples of this approach include prochelators that are activated by intracellular enzymes, under oxidative stress conditions, and by reduction of disulfide bonds.

The disulfide reduction/activation approach employs disulfide bonds as redox-sensitive switches that are triggered upon cell entry by the high concentrations of cytosolic thiols with respect to the extracellular milieu. For instance, the intracellular concentrations of reduced glutathione (5-11 mM) are orders of magnitude greater than those in the extracellular space and blood plasma (<5 μM). As such, disulfide linkages are employed extensively in prodrug and chemosensor design, and recent theranostic systems have allowed visualization of selective disulfide-based drug release in vivo. In addition, malignant tissue contains higher levels of glutathione when compared to the neighboring normal tissue, therefore disulfide switches are particularly attractive for the design of antiproliferative prochelator systems.

Inventors have previously shown that a disulfide bond can be employed to mask a sulfur donor within tridentate thiosemicarbazone chelators (see FIG. 2). The resulting disulfide-based prochelators do not coordinate metal ions in neutral aqueous solutions, whereas the thiolates generated upon intracellular reduction are high-affinity chelators. In the case of prochelator (TC1-S)₂ (FIG. 2), intracellular reduction and iron binding lead to the formation of a low-spin ferric complex that is not redox active. Inventors have also utilized disulfide switches as linkers in glycoconjugate prochelator systems targeting the overexpression of glucose transporters in colon cancer cells. Furthermore, disulfide-based thiosemicarbazone prochelators have the potential to alter iron trafficking and distribution in the tumor microenvironment through their effects on the iron metabolism of tumor-associated macrophages.

The present invention features a disulfide-based prochelation design to aroylhydrazone and semicarbazone scaffolds. The present invention also features the introduction of a methyl substituent at the iminic carbon in the new prototypes and in the original thiosemicarbazone framework. The present invention also describes intracellular effects of the corresponding chelators, which feature both (S, N, S) and (S, N, O) donor sets, with respect to redox behavior, cell cycle, toxicity and cell death.

Synthesis: The prochelators described herein fall into three classes of metal-binding scaffolds: the thiosemicarbazones (TC compounds), the aroyl hydrazones (AH compounds) and the semicarbazones (SC compounds). The selected compounds allowed comparison of binding groups with (S,N,S) and (S,N,O) donor sets, as well as different functional groups (e.g., aroyl hydrazones vs. semicarbazones within the (S,N,O) binding units). In addition, methyl hydrazone analogs were included because studies on SIH and related high-affinity hydrazone chelators indicated that these derivatives are more stable with respect to hydrolytic degradation.

Thioether TE1 was synthesized as a control analog of prochelator (TC1-S)₂ lacking the disulfide bond. TE1 does not bind iron in physiologically relevant conditions and cannot be reduced by intracellular thiols to produce an iron-binding species.

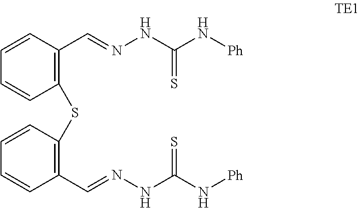 [SMILES: S=C(N/N=C/c1ccccc1Sc1ccccc1/C=N/NC(=S)Nc1ccccc1)Nc1ccccc1]

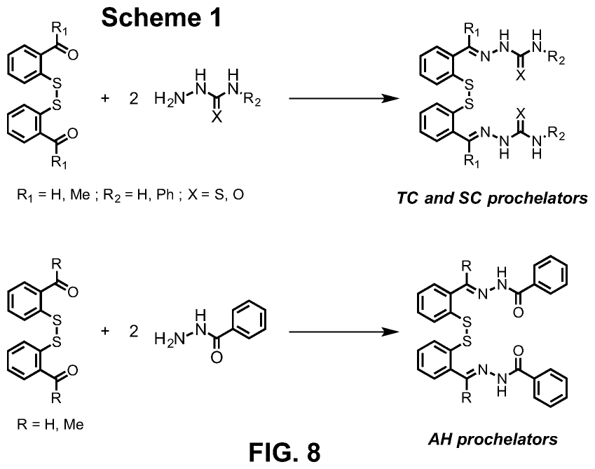

The disulfide-based prochelators were synthesized via Schiff-base condensation reactions between a 2,2′-dithiodibenzyl aldehyde or ketone and a thiosemicarbazide, semicarbazide, or aroyhydrazide (FIG. 8). Details are described below.

Without wishing to limit the present invention to any theory or mechanism, it is believed that an advantage in the purification of the compounds is the solubility difference between the starting materials and the products of the condensation reactions. Although the starting materials are soluble in refluxing alcohols (e.g., methanol, ethanol, isopropanol), the disulfide prochelators generally precipitate from the reaction mixtures.

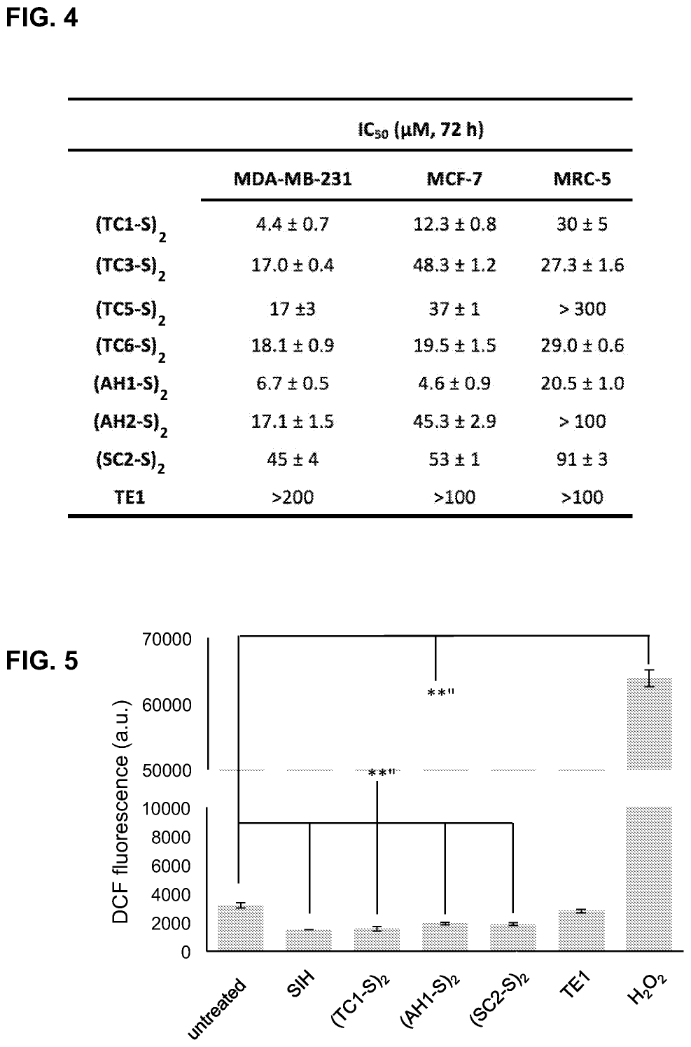

Antiproliferative activity: The assessment of the antiproliferative activity of the compounds focused on breast cancer because its association to an altered metabolism of iron is well documented. The prochelators were tested in breast adenocarcinomas cell lines MCF-7 (ATCC® HTB-22™) and MDA-MB-231 (ATCC® HTB-26™) as well as in the normal lung fibroblast cell line MRC-5 (ATCC® CCL-171™) using standard MTT assay protocols (FIG. 4).

The antiproliferative activity of the thiosemicarbazone and hydrazone-based disulfide-masked prochelators in cancer cell lines fall within a relatively narrow range, with IC₅₀ values mostly ranging from 4 to 50 μM. In the normal fibroblasts, which are typically less sensitive to iron sequestration and present a less reducing environment compared to malignant cells, IC₅₀ values were consistently higher than 20 μM and up to more than 100 μM for (AH2-S)₂ and (TC5-S)₂.

Within the tested panel, it was observed that the semicarbazone (SC2-S)₂ presented the highest IC₅₀ values. This observation is in line with previous studies showing that semicarbazones typically bind iron with lower affinity and have lower antiproliferative activity as compared to their thiosemicarbazone analogues. Confirming the requirement for intracellular activation and iron binding, the thioether control TE1 did not affect cell viability across all concentrations tested (up to 200 μM).

Inventors have previously shown that the exposure of cultured cells to prochelator (TC1-S)₂ leads to iron sequestration and intracellular formation of a low-spin Fe(III) complex. This ferric species is not susceptible to intracellular redox cycling and therefore does not elicit catalytic generation of reactive oxygen species (ROS).

Investigation of intracellular ROS generation: The ability of the disulfide prochelators to induce oxidative stress intracellularly was tested using 2′,7′-d ichlorodihydrofluorescein diacetate (DCFH₂-DA) as a fluorogenic probe. DCFH₂-DA, which is hydrolyzed to DCFH₂ and trapped intracellularly upon action of esterases, reacts with several ROS/RNS species (e.g., hydroxyl radical, peroxynitrite) to produce the fluorescent dichlorofluorescein (DCF) dye. Hydrogen peroxide (used as the positive control in this assay) does not react directly with DCFH₂, but its intracellular metal-mediated decomposition produces the detected ROS/RNS species.

Treatment of MDA-MB-231 breast adenocarcinoma cells with high-affinity chelator SIH (50 μM, 2 h) resulted in suppression of inherent ROS as demonstrated by a significant decrease of DCF fluorescence compared to the untreated control (FIG. 5). This behavior is well documented and SIH is in fact employed to protect cells against metal-mediated oxidative stress. For a selection of prochelators featuring one compound of each family of binding units, it was found that treatment of MDA-MB-231 cells (50 μM, 2 h) also results in a decrease of ROS/RNS levels compared to the untreated control. Conversely, treatment with thioether TE1, which lacks metal-binding affinity, does not result in suppression of basal ROS/RNS concentrations. The antioxidant behavior of the prochelators can therefore be ascribed to metal sequestration, which excludes Fe(II) ions from Fenton chemistry, rather than to radical/ROS scavenging reactivity by their organic framework.

Because the positive control (H₂O₂) showed a response that is an order of magnitude higher than that of the untreated control, it was concluded that at the tested concentrations and treatment time, the prochelators described herein in Example 1 do not result in induction of oxidative stress. Rather, as observed for SIH and several analogs, the disulfide-masked prochelators protect against metal-mediated intracellular oxidative stress as measured by DCFH₂-DA.

Effects on cell cycle progression: Iron chelation often causes cell cycle arrest at the G₁/S interface, as cells display lower DNA biosynthetic activity and cannot progress through the cell cycle. Although the decreased availability of iron affects several cellular processes, the G₁/S arrest has been attributed, at least in part, to the ability of chelators to decrease the activity of ribonucleotide reductase (RNR), an enzyme that is critical for DNA synthesis. Inventors have previously shown that treatment of cultured Jurkat lymphocyte cells with prochelator (TC1-S)₂ results in decreased intracellular levels of active RNR as measured by electron paramagnetic resonance (EPR) spectroscopy. The effects of a selection of prochelators of the present invention on cell cycle progression in the same cell line were investigated. As in the redox activity assays (vide supra), high-affinity chelator SIH was employed as a positive control.

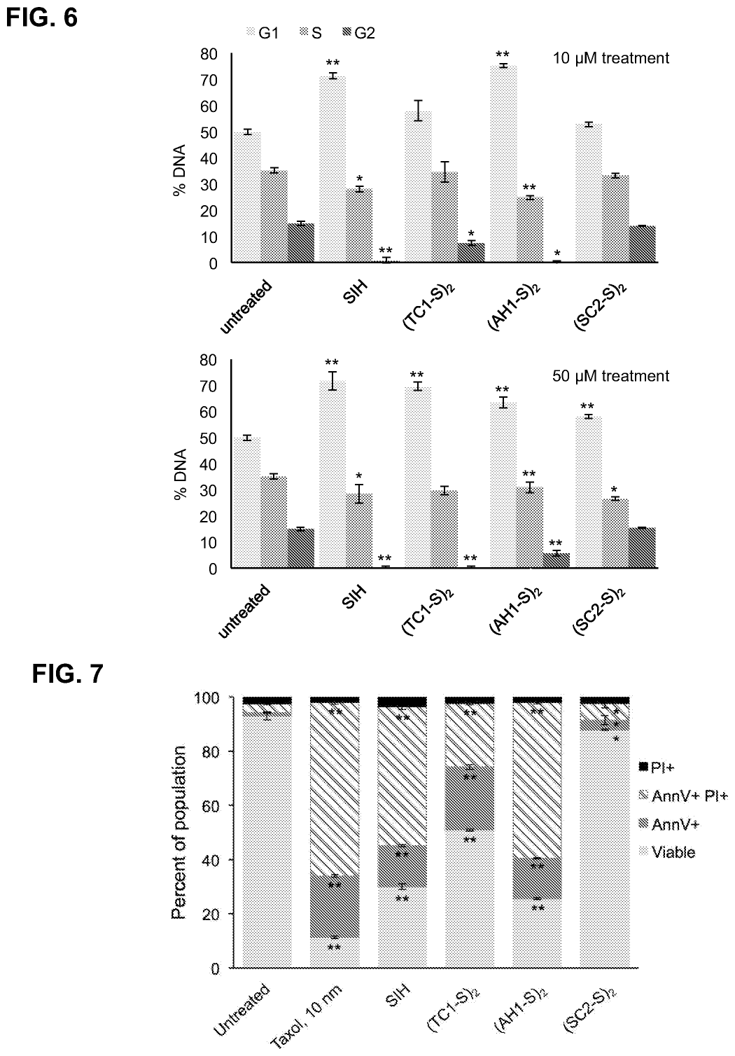

Cell cycle distributions in Jurkat cells were tested after 10 and 50 μM treatment and 12-hour incubations (FIG. 6). At 10 μM, SIH resulted in arrest at the G₁/S interface, with accumulation of G_{0/1} compared to the untreated control. At this concentration, treatment with (AH1-S)₂ resulted in G_{1/0} accumulation as significant as that for SIH. This accumulation of cells in the G_{1/0} phase is accompanied by statistically significant depletion of cells in the S phase, with (AH1-S)₂ being more effective than SIH (FIG. 6, top panel). Compound (SC1-S)₂ also led to significant accumulation of cells in G_{1/0} phase, whereas the other systems did not cause statistically significant changes in this assay. At higher concentrations (50 μM), however, all the prochelators have similar impact on cell cycle, with G_{1/0} accumulation and S depletion being statistically significant in all cases (FIG. 6, bottom panel). These data indicate that all the tested prochelators have effects on cell cycle that are consistent with iron sequestration.

Effects on cell death: Apoptosis is an important form of programmed cell death that is often implicated as the pathway of chelation-induced cytotoxicity. Iron chelators such as DFO, as well as several aroylhydrazones and thiosemicarbazones, initiate the apoptotic pathway of cell death. In the context of this analysis of the biological activity of disulfide-based prochelators, their effects on cell death were investigated using propidium iodide (PI) and a fluorescent analogue of Annexin V (AnnV) as probes for apoptotic markers.

The extent of apoptosis in Jurkat cells were assessed after treatment with prochelators of the present invention for 48 hours (FIG. 7). Potent antineoplastic agent taxol (paclitaxel) was used as a positive control known to induce apoptosis. Tridentate chelator SIH was included as a comparison leading to iron sequestration with no need for intracellular activation. For all tested prochelators, 48-hour treatments resulted in a decrease in viable cells compared to the untreated control. This observation is accompanied by a significant increase in populations that stain positive for AnnV only (termed apoptotic) or AnnV and PI (termed late-apoptotic). Within the prochelator series, (AH1-S)₂ was as effective as SIH at inducing apoptosis. The thiosemicarbazone-based prochelator (TC1-S)₂ is less effective and the semicarbazone analogue (SC1-S)₂ is the least potent of all the compounds tested. These results are consistent with the antiproliferative activity of this series of disulfide-masked prochelators as assessed by colorimetric assays as well as their efficacy in halting cell cycle progression (vide supra). For the thioether control compound TE1, which cannot be reductively activated to sequester intracellular iron, no statistically significant changes were observed in any of the populations (viable, apoptotic, etc.) compared to the untreated control, further validating the reduction/activation pathway as well as the necessity of iron scavenging to induce toxicity.

The induction of apoptosis in Jurkat cells by disulfide-masked prochelators is concentration- and time-dependent, and an increase in AnnV+/PI+ populations was observed with longer incubations or higher concentrations. None of the treatments resulted in significant populations that stained positive for PI only (less than 4% of the population in all cases), ruling out necrosis as a pathway of cell death. In fact, iron scavengers are known to suppress Fe-mediated necrosis that results from Fenton reactions and ROS generation. The lack of a significant necrotic population is in line with the fact that prochelators of the present invention suppress basal ROS levels (vide supra).

Collectively, and in agreement with the IC₅₀ values (FIG. 4), the data from cell cycle and cell death assays indicate that aroyl hydrazone (AH1-S)₂ and thiosemicarbazone (TC1-S)₂ are more effective than semicarbazone (SC1-S)₂ as antiproliferative prochelators.

Synthesis of disulfide prochelators: (TC1-S)₂, and (TC3-S)₂ were synthesized according to reported procedures. The other prochelators were synthesized via condensation of a thiosemicarbazide, semicarbazide, or hydrazide with a 2,2′-dithiodibenzyl dialdehyde or methyldiketone (see FIG. 8).

For example, (TC5-S)₂ was synthesized as follows: 1′-(disulfanediylbis(2,1-phenylene))diethanone (150 mg, 0.49 mmol) was suspended in ethanolic HCl (4 mL, 0.12 mM) and was heated to reflux for 30 minutes and 4-phenyl thiosemicarbazide (150 mg, 0.86 mmol) was then added as a solid. The reaction mixture was allowed to reflux for 3 hours. Upon cooling, the resulting precipitate was isolated by filtration, washed with water and dried under vacuum (152 mg, 77% yield). ¹H NMR (500 MHz, DMSO-d₆) δ 11.05 (s, 1H), 10.03 (s, 1H), 7.82 (dd, J=8.1, 1.1 Hz, 1H), 7.70 (ddd, J=9.8, 8.1, 1.1 Hz, 3H), 7.40 (dtd, J=32.2, 7.4, 1.4 Hz, 3H), 7.30-7.23 (m, 2H), 7.13 (tt, J=7.0, 1.1 Hz, 1H), 2.48 (s, 3H). ¹³C NMR (126 MHz, DMSO-d₆) δ 176.42, 149.39, 139.08, 137.44, 135.38, 130.20, 129.802, 128.734, 127.04, 126.79, 125.35, 123.69, 17.11. HRMS m/z [M+Na]⁺ calculated for C₃₀H₂₈N₆S₄Na 623.11560; found, 623.11488.

(TC6-S)₂ was synthesized as follows: 1′-(disulfanediylbis(2,1-phenylene))diethanone (150 mg, 0.49 mmol) was suspended in ethanolic HCl (7.5 mL, 0.12 mM) and heated to reflux for 30 minutes. Thiosemicarbazide (180 mg, 1.97 mmol) was then added to the reaction flask as a solid and the mixture was allowed to reflux for 3 hours. Upon cooling to room temperature, the precipitate was isolated by filtration, washed with water then dried under vacuum (210 mg, 95% yield). ¹H NMR (500 MHz, DMSO-d₆) δ 10.62 (s, 1H), 8.43 (s, 1H), 7.72-7.67 (m, 1H), 7.64 (dd, J=7.7, 1.3 Hz, 1H), 7.56-7.51 (m, 1H), 7.36 (dtd, J=29.9, 7.6, 1.2 Hz, 2H), 2.40 (s, 3H). ¹³C NMR (126 MHz, DMSO-d₆) δ 179.59, 148.93, 137.63, 135.37, 130.01, 129.63, 126.95, 126.86, 17.05. HRMS m/z [M+Na]⁺calculated for C₁₈H₂₀N₆S₄Na, 471.05300; found, 471.05281.

(AH1-S)₂ was synthesized as follows: 2,2′-dithiobenzyldialdehyde (235 mg, 0.86 mmol) was combined with benzhydrazide (256 mg, 1.88 mmol) in ethanol (5 mL) and brought to reflux to dissolve the starting materials. The reaction progress was monitored by TLC. Upon completion, a white solid was collected by filtration, washed with ethanol (3×6 mL) and dried under vacuum (333 mg, 76%). ¹H NMR (500 MHz, DMSO-d₆) δ 12.07 (s, 1H), 8.92 (s, 1H), 7.96 (d, J=7.4 Hz, 2H), 7.89 (d, J=6.9 Hz, 1H), 7.65 (dd, J=27.8, 7.3 Hz, 2H), 7.56 (t, J=7.5 Hz, 3H), 7.43 (p, J=7.0 Hz, 2H). ¹³C NMR (126 MHz, DMSO-d₆) δ 163.49, 145.90, 135.74, 134.02, 133.66, 132.35, 130.88, 129.86, 128.97, 128.78, 128.45, 128.14, 110.01. HRMS m/z [M+H]⁺calculated for C₂₈H₂₃N₄O₂S₂, 511.12624; found 511.12565; m/z [M+Na]⁺ calculated for C₂₈H₂₂N₄O₂S₂Na, 533.10819; found 533.10752.

(AH2-S)₂ was synthesized as follows: 1′-(disulfanediylbis(2,1-phenylene))diethanone (37 mg, 0.12 mmol) was suspended in ethanolic HCl (5 mL, 0.12 mM) and was heated to reflux for 30 minutes to dissolve the starting material. Benzhydrazide (42 mg, 0.31 mmol) was then added to the reaction flask as a solid and the solution was allowed to reflux for 3 hours. The resulting precipitate was isolated by vacuum filtration, washed with water, and then dried under vacuum (35 mg, 53% yield). ¹H NMR (500 MHz, DMSO-d₆) δ 9.76 (s, 1H), 8.16 (dd, J=7.8, 1.4 Hz, 1H), 7.82 (dd, J=8.3, 1.4 Hz, 1H), 7.65 (dd, J=8.2, 1.1 Hz, 1 H), 7.59-7.55 (m, 1 H), 7.55-7.48 (m, 1 H), 7.45 (ddt, J=8.2, 6.7, 1.2 Hz, 1 H), 7.41 (ddd, J=7.7, 7.3, 1.2 Hz, 2H), 2.70 (s, 3H). ¹³C NMR (126 MHz, DMSO-d₆) δ 200.03, 166.40, 138.96, 134.73, 133.83, 132.93, 131.49, 128.74, 127.38, 126.45, 125.81, 28.01. HRMS m/z [M+H]⁺calculated for C₃₀H₂₇N₄O₂S₂, 539.15754; found 539.15801; m/z [M+Na]⁺ calculated for C₃₀H₂₆N₄O₂S₂Na, 561.13949; found 561.13960.

(SC1-S)₂ was synthesized as follows: 2,2′-dithiobenzyldialdehyde (267 mg, 0.97 mmol) was combined with semicarbazide hydrochloride (325 mg, 2.92 mmol) in ethanol (2 mL) and brought to reflux to dissolve the starting materials. The reaction progress was monitored by TLC. Upon completion, an off-white solid was collected by filtration, washed with ethanol (3×4 mL) and dried under vacuum (322 mg, 98%). ¹H NMR (500 MHz, DMSO-d₆) δ 10.49 (s, 1H), 8.30 (s, 1 H), 7.96-7.90 (m, 1H), 7.62-7.56 (m, 1 H), 7.38-7.32 (m, 2H), 6.48 (s, 2H). ¹³C NMR (126 MHz, DMSO-d₆) δ 156.86, 137.62, 134.24, 129.91, 129.55, 128.60, 128.33. HRMS m/z [M+Na]⁺calculated for C₁₆H₁₆N₆O₂S₂Na, 411.06738; found 411.06697.

(SC2-S)₂ was synthesized as follows: 1′-(disulfanediylbis(2,1-phenylene)) diethanone (100 mg, 0.33 mmol) was combined with semicarbazide hydrochloride (110 mg, 0.99 mmol) in ethanol (2 mL) and brought to reflux. The reaction progress was monitored by TLC. An off-white product was collected by filtration, washed with ethanol (3×4 mL) and dried under vacuum (20 mg, 15%). ¹H NMR (500 MHz, DMSO-d₆) δ 9.65 (s, 1 H), 7.65 (dd, J=8.0, 1.3 Hz, 1 H), 7.56 (dd, J=7.6, 1.5 Hz, 1 H), 7.36-7.26 (m, 2H), 6.40 (s, 2H), 2.28 (s, 3H). ¹³C NMR (126 MHz, DMSO-d₆) δ 157.35, 144.98, 138.04, 129.36, 129.05, 126.75, 126.53. HRMS m/z [M+H]⁺calculated for C₁₈H₂₁N₆O₂S₂, 417.11674; found 417.11630; m/z [M+Na]⁺ calculated for C₁₈H₂₀N₆O₂S₂Na, 439.09868; found 439.09826.

Cytotoxicity assays: MTT (3-(4,5-dimethylthiazol-2-yl)-2,5-diphenyltetrazolium bromide) viability assays were conducted as previously described.²² Cells were seeded at 4000 cells per well for MCF-7, at 10,000 cells per well for MRC-5 or at 700 cells per well for MDA-MB-231 and allowed to attach for 24 h. Test compounds dissolved in DMSO were diluted in EMEM to the specified concentration with final DMSO concentration limited to 0.1% v/v. Cells were incubated in the presence of the test compounds for 72 h, then the MTT solution (4 mg/mL, 10 μL) was added to each well and incubated for 4 h. Following media removal, DMSO (100 μL) was added to each well to dissolve the purple formazan crystals and the plates were incubated for an additional 30 minutes. Absorption at 560 nm was recorded and data were analyzed using logarithmic fits (Origin®) to obtain IC₅₀ values. Each experiment was conducted in triplicate, and values are given as mean ±SDM.

Detection of Intracellular ROS: Solutions of the fluorescent probe DCFH₂-DA (Invitrogen) were prepared in DMSO, aliquoted in single-use doses and stored at −20° C. MDA-MB-231 cells were seeded in 6-well plates at a density of 1.0×10⁵ cells/mL (2.0×10⁵ cells/well) and allowed to incubate overnight. The growth media were then removed, and the adherent cells were treated for 2 hours with the test compounds in phenol-red free EMEM (0.1% DMSO was used to solubilize the compounds). The positive control (H₂O₂) was diluted in PBS and cells were exposed to this solution for 10 minutes. After incubation, the cells were washed with PBS and then treated with warm PBS containing DCFH₂-DA (30 μM) for 20 min. After removal of the probe solution, the cells were washed with PBS (×2) and detached using trypsin-EDTA (3 min). The cell suspension was diluted with growth media (2 mL) and centrifuged at 2000 rpm for 10 minutes. The resulting cell pellet was then suspended in PBS (500 μL), stored on ice, and analyzed by flow cytometry within one hour.

Cell cycle analysis: MDA-MB-231 cells were seeded at 0.2 million cells per well (in EMEM supplemented with 0.1 mg/mL human holo-transferrin) in 6-well plates and allowed to adhere overnight. Media were removed and solutions containing test compounds were added (final DMSO concentration 0.1% v/v) and cells were incubated for the specified time. Media were then collected, cells washed with PBS (1 mL) and then detached by addition of 0.25% trypsin-EDTA (0.4 mL) followed by a 3-minute incubation. After addition of EMEM (1 mL), the cell suspension was centrifuged at 125 xg for 15 minutes. Media were discarded, and cells were fixed by addition of ice-cold 70% ethanol (2 mL) and stored in a freezer at −20° C. overnight (and no longer than one week). Cells were spun at 2000 rpm for 20 minutes, and the resulting pellet was suspended in PBS (0.3 mL) and treated with RNAse and propidium iodide (0.5 mg/mL and 40 μg/mL respectively, 30 min), placed on ice and analyzed by flow cytometry within one hour.

Apoptosis assays: Jurkat cells were grown to 1.5 million cells/mL, then centrifuged and suspended in fresh RPMI medium at 0.5 million cells/mL. Cells were then treated with either test compounds dissolved in DMSO or DMSO alone for control samples (final DMSO concentration 0.1% v/v). Cells were incubated for the specified time and then aliquots of 1 million cells were centrifuged. The pellets were suspended in binding buffer (10 mM HEPES with 150 mM NaCl, 5 mM KCl, 1 mM MgCl₂, and 1.8 mM CaCl₂, pH 7.4) containing 2μg/mL FITC-Annexin V (0.3 mL) and solutions were incubated in the dark at room temperature for 10 minutes. Propidium iodide was added prior to analysis at a final concentration of 1 μg/mL.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1. A pro-chelator comprising at least one pro-ligand and a disulfide bond, wherein the disulfide bond is connected to the pro-ligand, and wherein each pro-ligand comprises at least two donor atoms.

2. The pro-chelator of claim 1, wherein the pro-chelator comprises two pro-ligands, connected by the disulfide bond.

3. The pro-chelator of claim 1, wherein the pro-chelator comprises one pro-ligand and a solubilizing or biologically active moiety, connected by the disulfide bond.

4. The pro-chelator of claim 1, wherein the pro-chelator is activated to transform each pro-ligand to an active bidentate, tridentate, or polydentate chelator.

5. The pro-chelator of claim 4, wherein the pro-chelator is activated by reduction of the disulfide bond.

6. The pro-chelator of claim 4, wherein each active chelator comprises a semicarbazone, a thiosemicarbazone, a hydrazone, or a thiohydrazone moiety.

7. The pro-chelator of claim 4, wherein each active chelator comprises an iminic position, and comprises an electron-withdrawing group at the iminic position.

8. The pro-chelator of claim 4, wherein each active chelator is configured to coordinate Fe to form a metal complex.

9. The pro-chelator of claim 8, wherein the metal complex has a Fe^{III}/Fe^{II} potential of about −200 to 200 mV compared to a Normal Hydrogen Electrode (NHE).

10. The pro-chelator of claim 1, wherein the pro-chelator comprises a structure according to Formula II, Formula III, Formula IV, or Formula V; 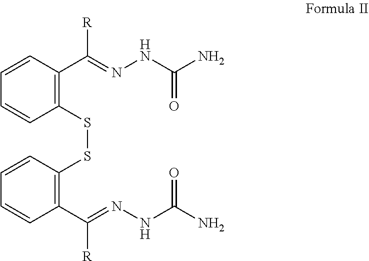
wherein R is H, alkyl, trifluoromethyl, aryl, or a derivative thereof; 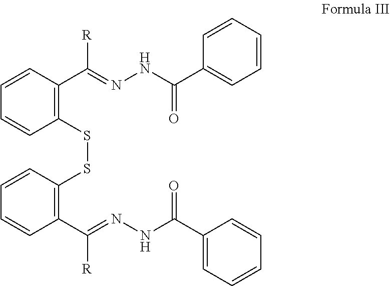
wherein R is H, alkyl, aryl or a derivative thereof; 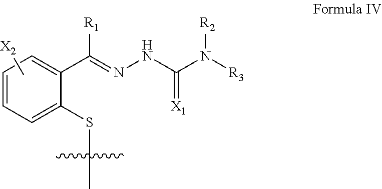
wherein:
if X₁=O
then R₁=Ph, pyridyl, p-CF₃-Ph, p-NO₂-Ph CCl₃ or CF₃;
X₂=H, alkyl, alkoxy, halo, CF₃ or NO₂;
R₂=H, alkyl, aryl, or substituted aryl; and
R₃=H, alkyl, aryl, or substituted aryl;
or if X₁=S
then R₁=Ph, pyridyl, p-CF₃-Ph, p-NO₂-Ph CCl₃ or CF₃;
X₂=H, alkyl, alkoxy, halo, CF₃ or NO₂;
R₂=alkyl, aryl, or substituted aryl; and
R₃=alkyl, aryl, or substituted aryl; 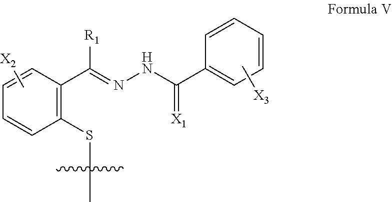
wherein: X₁=O, or S;
R₁=Ph, pyridyl, p-CF₃-Ph, p-NO₂-Ph CCl₃ or CF3^{.}
X₂=H, alkyl, alkoxy, halo, CF₃ or NO₂; and
X₃=H, alkyl, alkoxy, halo, CF₃ or NO₂.

11. A method of preventing iron-deficiency anemia while treating a subject having malignant cells characterized by a reprogrammed iron metabolism, the method comprising:
a. providing a pro-chelator to a bloodstream of the subject, the pro-chelator comprising at least one pro-ligand and a disulfide bond, wherein the disulfide bond is connected to the pro-ligands, and wherein each pro-ligand comprises at least two donor atoms; and
b. transporting the pro-chelator to an intracellular space of a malignant cell of the subject;
wherein an active chelator is selectively released from the pro-chelator by reduction of the disulfide bond within the intracellular space,
wherein the active chelator coordinates Fe selectively within the intracellular space to form a metal complex, and does not coordinate Fe in the bloodstream of the subject, and
whereby the selective coordination of Fe is effective for treating the malignant cells without causing iron-deficiency anemia.

12. The method of claim 11, wherein the pro-chelator comprises two pro-ligands, connected by the disulfide bond.

13. The method of claim 11, wherein the pro-chelator comprises one pro-ligand and a solubilizing or biologically active moiety, connected by the disulfide bond.

14. The method of claim 11, wherein the active chelator comprises a semicarbazone, a thiosemicarbazone, a hydrazone, or a thiohydrazone moiety.

15. The method of claim 11, wherein the metal complex has a Fe^{III}/Fe^{II} potential of about −200 to 200 mV compared to a Normal Hydrogen Electrode (NHE).

16. The method of claim 11, wherein the active chelator comprises an iminic position, and comprises an electron-withdrawing group at the iminic position.

17. The method of claim 11, wherein the active chelator is bidentate, tridentate, or polydentate.

18. The method of claim 11, wherein the pro-chelator comprises a structure according to Formula II, Formula III, Formula IV, or Formula V; 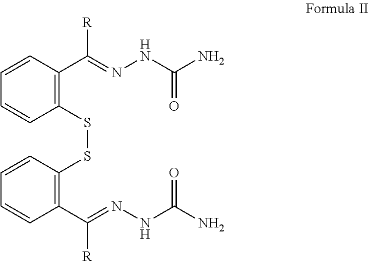
wherein R is H, alkyl, trifluoromethyl, aryl, or a derivative thereof; 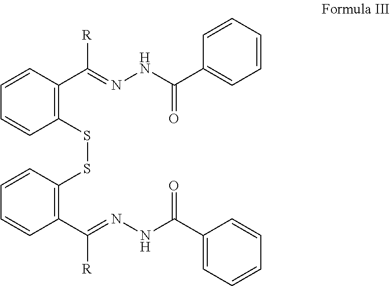
wherein R is H, alkyl, aryl or a derivative thereof; 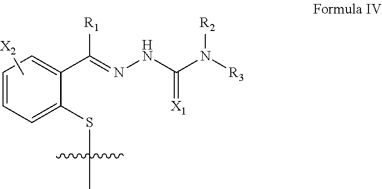
wherein:
if X₁=O
then R₁=Ph, pyridyl, p-CF₃-Ph, p-NO₂-Ph CCl₃ or CF₃;
X₂=H, alkyl, alkoxy, halo, CF₃ or NO₂;.
R₂=H, alkyl, aryl, or substituted aryl; and
R₃=H, alkyl, aryl, or substituted aryl;
or if X₁=S
then R₁=Ph, pyridyl, p-CF₃-Ph, p-NO₂-Ph CCl₃, or CF₃;
X₂=H, alkyl, alkoxy, halo, CF₃ or NO₂;.
R₂=alkyl, aryl, or substituted aryl; and
R₃=alkyl, aryl, or substituted aryl; 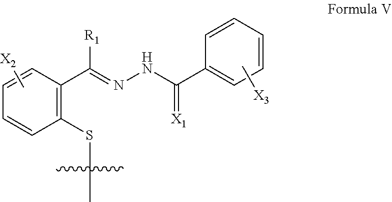
wherein: X₁=O, or S;
R₁=Ph, pyridyl, p-CF₃-Ph, p-NO₂-Ph CCl₃ or CF3;
X₂=H, alkyl, alkoxy, halo, CF₃ or NO₂; and
X₃=H, alkyl, alkoxy, halo, CF₃ or NO₂.

19-22. (canceled)

23. A pro-chelator according to Formula III, wherein R is H, alkyl, aryl or a derivative thereof.

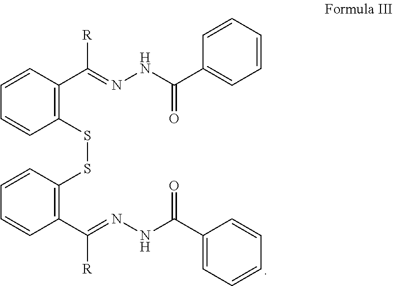

24. The pro-chelator of claim 23, wherein the pro-chelator is redox-activated.

25-34. (canceled)
